(19) United States
(12) Patent Application Publication
(10) Pub. No.: US 2013/0079531 A1
(43) Pub. Date: Mar. 28, 2013

(54) Process for the Rapid Extraction of Active Ingredients from Herbal Materials

(71) Applicant: Rm3 Labs LLC, Boulder, CO (US)

(72) Inventor: Ian Barringer, Boulder, CO (US)

(73) Assignee: Rm3 Labs LLC, Boulder, CO (US)

(21) Appl. No.: 13/629,438

(22) Filed: Sep. 27, 2012

Related U.S. Application Data
(60) Provisional application No. 61/539,476, filed on Sep. 27, 2011.

(51) Int. Cl. C07D 311/78

(52) U.S. Cl. CPC C07D 311/78
USPC 549/390; 422/255

(57) ABSTRACT

The invention is a process for the rapid extraction of active ingredients from herbal materials using a cold solvent and a very short mixing period in order to yield commercially desirable extracts. In particular, the process can be applied to the rapid extraction of cannabinoids from cannabis. The claimed invention also includes any equipment or machine, or assemblage of equipments or machines, designed or employed to utilize this process.

### CROSS-REFERENCE TO RELATED APPLICATION

This application claims benefit of Provisional Patent Application No. 61/53.9476, “A Process for the Rapid Extraction of Cannabinoids from Cannabis”, filed Sep. 27, 2011.

### FIELD OF THE INVENTION

This invention is a process for the rapid extraction of active ingredients from herbal materials to produce commercially desirable extracts. In particular, the process can be applied to the rapid extraction of cannabinoids from cannabis.

### STATEMENT REGARDING FEDERALLY SPONSORED RESEARCH OR DEVELOPMENT

Not applicable.

### BACKGROUND OF THE INVENTION

Recent innovations in the field of cannabinoid extraction from cannabis materials have largely been directed towards the creation of cannabinoid extracts of high purity, often with cannabinoid levels in end materials in excess of 95% by weight. Often isolation of a single cannabinoid, of the more than 80 identified natural cannabinoids, is desired.

Many of the developments have focused on the use of liquid or supercritical carbon dioxide as a solvent, either alone or with one or more co-solvents. See, for example, Elsohly et al. (2004), U.S. Pat. No. 6,730,519; Flockhart et al. (2010) U.S. Pat. No. 7,700,368. These production methods are largely directed towards inclusion of the resulting cannabinoid preparations in pharmaceutical products.

Such production methods tend to be slow, requiring extraction times measured in hours, and, in the case of supercritical carbon dioxide extraction, capital intensive due to the high pressures involved. In addition, significant amounts of active ingredients may be left behind in the extracted material. These limitations make these technologies difficult and/or expensive to scale.

By contrast, the growth of the medical marijuana market in the United States and in other nations has led to demand for lower-cost extraction methods. For many medical marijuana preparations, the retention of the full mix of cannabinoids present in the cannabinoid-bearing material is desirable. In addition, it may be desirable to retain certain of the terpenoid or phenolic compounds found in marijuana, which give the product is characteristic smell and which, in many cases, themselves have physiological activity.

Given prior-generation technologies—ethanol extraction or butane extraction followed by evaporation, or mechanical separation such as “kiefing” or “bubble hashing”—purities of 35% to 55% total cannabinoid content by weight can readily be achieved. Carbon dioxide extraction can yield purities in excess of 70% and, when specifically tailored or when subsequent processing is done, purities in excess of 90% are possible. However, most of these technologies leave behind significant levels—often a third—of available cannabinoids in the extracted material. The most efficient of these methods, ethanol extraction, when carefully performed can extract 95% of available cannabinoids. However, the purity of the evaporated ethanol product is frequently low, often less than 35%, due to the solvent's tendency to extract unwanted products together with the cannabinoids. This tendency is exacerbated by the extraction period of days or weeks traditionally used in the formulation of ethanol-based cannabis tinctures.

The present invention is a novel process which allows for the rapid extraction of active ingredients in herbal materials. In particular, the process can extract nearly all cannabinoids in cannabis in minutes. Depending on the desired outcome and the quality of the cannabinoid-bearing material, extracts containing in excess of 40 milligrams of cannabinoids per milliliter can be obtained. When evaporated, such extracts can yield an end product containing in excess of 60% total cannabinoids, with fewer objectionable tastes and smells than traditionally prepared alternatives.

### BRIEF SUMMARY OF THE INVENTION

The claimed invention is a process for the extraction of active ingredients from plant or other materials using a cold solvent and a very short mixing period in order to yield commercially desirable extracts. In particular, the process can be applied to the rapid extraction of cannabinoids from cannabis. This process takes advantage of the fact that (1) the majority of cannabinoids in raw plant materials is found on the surface of the material and (2) the solubility of cannabinoids in most solvents remains relatively stable as the reaction temperature drops from 20 degrees centigrade to minus 20 degrees centigrade, while the solubility of other materials, particularly chlorophyll and similar molecules, is reduced greatly as the temperature drops. The claimed invention also includes any equipment or machine, or assemblage of equipments or machines, designed or employed to utilize this process.

### BRIEF DESCRIPTION OF DRAWINGS

FIG. 1: Process schematic diagram.

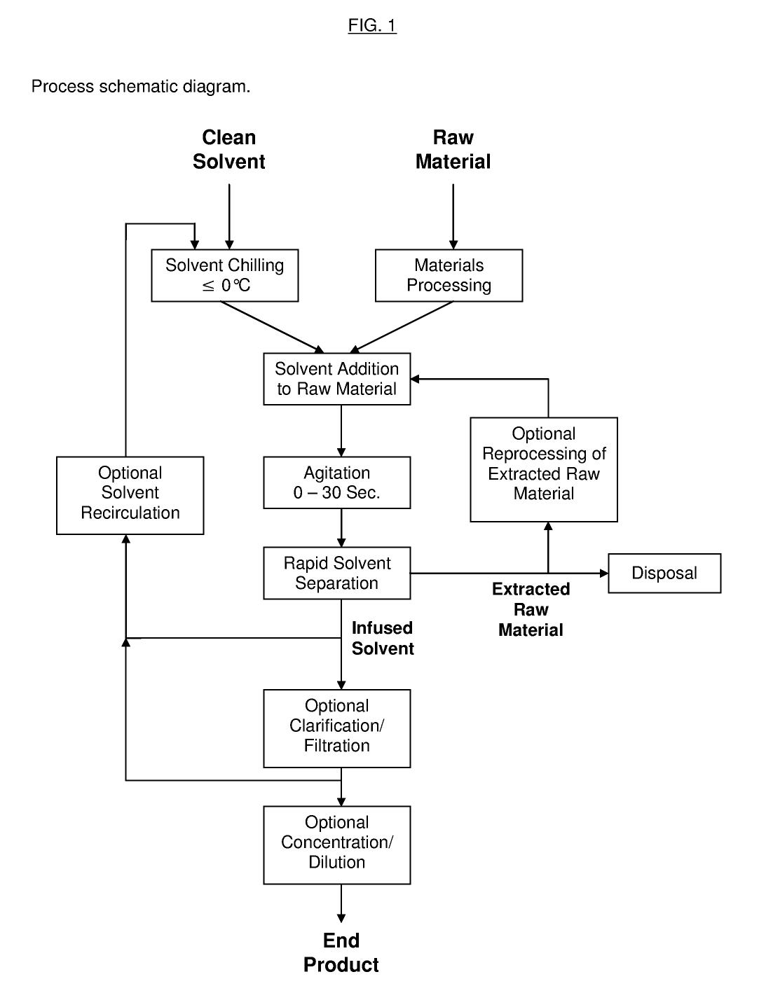

FIG. 2: Rate of extraction of THC vs. extraction of pigments (including chlorophylls and carotenoids) at 20° C. and −12° C.

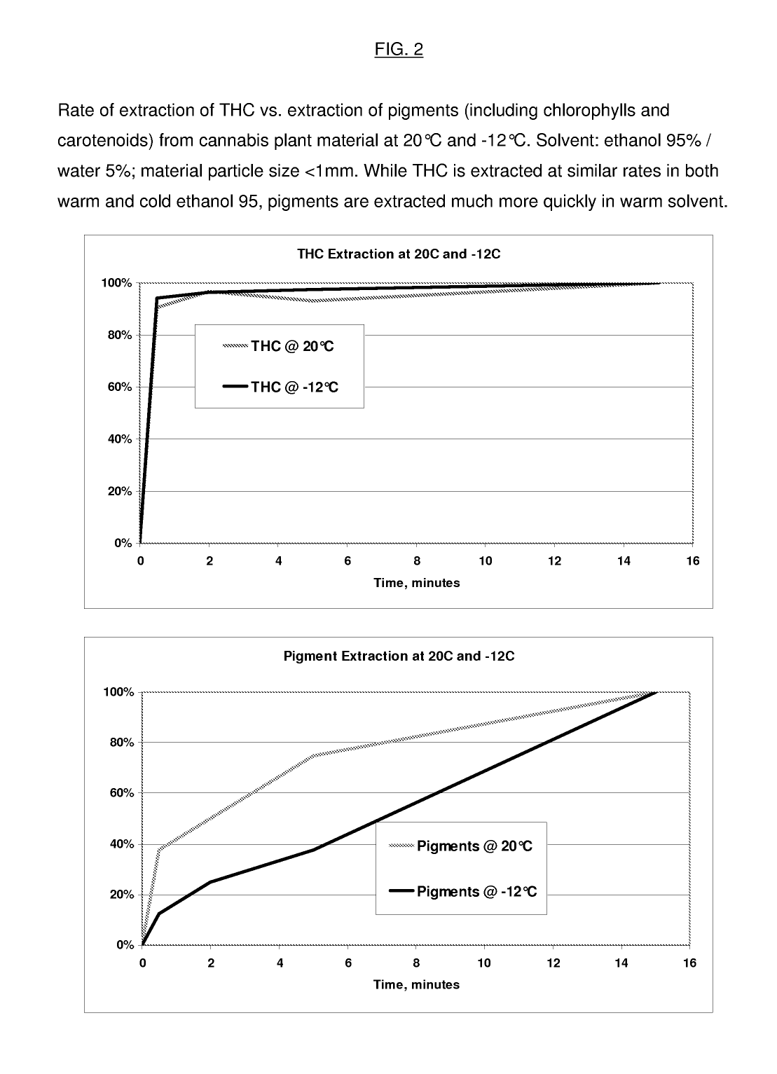

### DETAILED DESCRIPTION OF THE INVENTION

The claimed invention is a process for the extraction of active ingredients from plant or other materials using a cold solvent and a very short mixing period in order to yield commercially desirable extracts. In particular, the process can be applied to the rapid extraction of cannabinoids from cannabis. This method takes advantage of the fact that (1) the majority of cannabinoids in raw plant materials is found on the surface of the material and (2) our research has determined that the solubility of cannabinoids in most solvents remains relatively stable as the reaction temperature drops from 20 degrees centigrade to minus 20 degrees centigrade, while the solubility of other materials, particularly chlorophyll and similar molecules, is reduced greatly as the temperature drops. The invention also includes any equipment or machine, or assemblage of equipment or machines, designed or employed to utilize the processes described herein.

Freshly harvested or dried plant material may be used as a source material for this process. Certain low-grade cannabis extracts, such as inferior grades of kief, and other cannabinoid-bearing materials may also be processed with this method.

To the extent desirable, the herbal material is first processed to remove stems, stalks, seeds or other material other than leaves and flowering buds. The exclusion of such material will generally yield a product with higher active ingredient levels. Such processing should generally be minimized in order to avoid breakage of cellular membranes, which will increase the level of non-active compounds, particularly chlorophyll, in the end product.

In the preferred embodiment, ethanol (ethyl alcohol) or ethanol 95%-water 5% is used as the extraction solvent. In order to minimize the extraction of chlorophyll and other undesired compounds, the solvent is kept cold—preferably below 0° C.—and contact between the herbal material and the solvent is ideally limited to a few minutes or less. The combined herbal material and solvent may be agitated during this period. Such agitation can be accomplished through the natural action of the setup employed, such as the turbulent passage of the solvent through stationary material, the movement of the herbal material/solvent mixture through a feed auger, or through such nondisruptive methods as shaking or low-intensity ultrasound.

The solvent and herbal material are generally combined either just before or simultaneous with the entry of the plant material into the separating machinery. In one embodiment, the solvent may be introduced to the plant material in an auger conveying the plant material from a containment bin to the separating machinery. In another embodiment, the solvent may be sprayed on the herbal material as the material enters a centrifugal separator or belt press. In either case, the time between combination and separation (the “latency period”) is carefully controlled.

Temperature must also be controlled during the latency period. Accordingly, the solvent is generally chilled before it is introduced to the herbal material. In one embodiment, the solvent is chilled below 0° C. In the most preferred embodiment, the solvent is chilled below −10° C. Other elements coming in contact with the combined herbal material and solvent may also be chilled. The herbal material can also be cooled prior to extraction; however, freshly harvested material should not be frozen to avoid disruption of cellular membranes.

After the desired latency period, the solvent is then quickly separated from the herbal material through pressing, centrifugal screening, expulsion by compressed air or a similar process. As the length of the latency period must be carefully controlled, this invention is best embodied by the use of a continuous or semi-continuous process for separating the solvent, such as through use of a pusher or scraper centrifuge, belt press or similar machinery. Batch processing may also be used, for example by passing the solvent through an extraction chamber filled with compacted herbal material or by filtering the herbal material out of the mixture.

Contact between the herbal material and the solvent, with proper materials preparation, agitation and/or architecture, can remove 90% or more of the active ingredients from the herbal material in 30 seconds.

The extraction may be repeated in order to achieve extraction of 95% to 99% of the starting active ingredients in the herbal material. This second pass will generally produce tinctures of lower strength, and evaporated product of lower purity, than the first pass.

In a further embodiment, the infused solvent may then be filtered or clarified to remove any remaining suspended material from the solvent.

In a further embodiment, part or all of the infused solvent may be recirculated and introduced to further herbal material, increasing the active ingredient content of the final extract.

The infused solvent can be used in the form in which it emerges from the separation process. Alternatively, it can be diluted or concentrated to a particular desired strength or mixed with other ingredients, or the solvent may be evaporated entirely to leave a material comprised primarily of active ingredients.

The solvent may be any non-polar or mildly polar solvent. Embodiments include butane, hexane, cyclohexane, ethane, pentane, octane, diethyl ether, methanol, ethanol, isopropanol, n-propanol, chloroform, ethyl acetate, acetone, diethylamine, xylene, dioxane or similar hydrocarbons or alcohols. However, toxicity and overall safety must be considered in the selection of the solvent. Preferred solvents include hexane and pentane, due to their low polarity and relative safety. In the most preferred embodiment, the solvent is 95% ethanol/5% water, due to its ready availability, significant cannabinoid carrying capacity and relative safety. While the 95% ethanol mix is more polar than many of the alternatives, which raises the possibility of extracting significant levels of ballast such as chlorophyll, the low temperature and fast extraction of this process significantly reduce uptake of these undesired substances.

The embodiments of the invention in which an exclusive property or privilege is claimed are defined as follows:

1. A method for the extraction of active ingredients from herbal material comprising: (i) introducing the herbal material to a non-polar or mildly polar solvent at or below a temperature of 10 degrees centigrade and (ii) rapidly separating the herbal material from the solvent after a latency period not to exceed 15 minutes.

2. The process defined in claim 1 in which the herbal material is cannabis.

3. The process defined in claim 1 in which the combined herbal material and solvent are agitated briefly prior to the separation of the herbal material and the solvent.

4. The process defined in claim 1 in which the solvent, the mixture, the herbal material and/or all or a portion of the extraction equipment is maintained below 10 degrees centigrade throughout the extraction process.

5. The process defined in claim 1 in which the solvent is chilled to zero degrees centigrade or below prior to its introduction to the herbal material.

6. The process defined in claim 1 in which the time from the introduction of the solvent to the separation of the mixture is less than two minutes.

7. The process defined in claim 1 in which the separation of the solvent from the herbal material is achieved through a continuous or semi-continuous process, such as through the use of a basket centrifuge, pusher centrifuge, decanter centrifuge, screen centrifuge, inverting bag centrifuge, conveyor belt filter, horizontal vacuum filter, belt press or similar apparatus.

8. The process defined in claim 1 in which the separation of the solvent from the herbal material is achieved through a batch process, such as in a screw press or by running the mixture through a stationary filter or screen.

9. The process defined in claim 1 in which the extraction process is repeated after the herbal material is separated from the solvent to extract further active ingredients from the herbal material.

10. The process defined in claim 1 in which part or all of the infused solvent from the extraction process is ‘recycled’ and introduced to further herbal material.

11. The process defined in claim 1 in which the infused solvent is diluted, concentrated or mixed with other ingredients to yield a commercially desirable product.

12. Any equipment or machine, or assemblage of equipment or machines, designed or employed to utilize the process defined in claim 1.
